(19) United States
(12) Patent Application Publication
(10) Pub. No.: US 2026/0316079 A1
(43) Pub. Date: Oct. 15, 2026

(54) ISOELECTRIC FOCUSING DEVICES AND METHODS

(71) Applicant: INTABIO, LLC, FREMONT, CA (US)

(72) Inventor: Wesley CHANG, FREMONT, CA (US)

(21) Appl. No.: 19/485,339

(22) PCT Filed: May 14, 2024
(86) PCT No.: PCT/US2024/029187

§ 371 Date: Nov. 18, 2025

Related U.S. Application Data
(60) Provisional application No. 63/504,119, filed on May 24, 2023.

(51) Int. Cl. B01L 3/00; C07K 1/28; G01N 27/447

(52) U.S. Cl. CPC B01L 3/502776; B01L 3/502715; B01L 3/502761; C07K 1/28; G01N 27/44791; G01N 27/44795; B01L 2200/027; B01L 2200/0636; B01L 2200/0647; B01L 2300/0816; B01L 2300/0864

(57) ABSTRACT

Disclosed and claimed are methods, devices, and systems that enable improved quantitative performance for the separation and analysis of analytes in an analyte mixture. In particular, narrowing of the width of a tributary channel right where it junctions with the separation channel results in a junction geometry that prevents the spatial distortion of a transiting focused sample.

### RELATED APPLICATIONS

The present patent application claims the priority benefit of U.S. Provisional Patent Application Ser. No. 63/504,119, filed May 24, 2023, the content of which is hereby incorporated by reference in its entirety into this disclosure.

### BACKGROUND

In isoelectric focusing, a sample, such as a protein sample, is mixed with an ampholyte solution. This sample/ampholyte solution is then transferred into a channel (e.g., a separation channel in a fluidic device, a capillary, or a capillary channel). One end of the channel is then connected to an anolyte solution (e.g., an acidic solution) and the other end is connected to a catholyte solution (e.g., a basic solution). A high voltage is applied across the channel, causing ion migration and, as a result, establishing a pH gradient across the channel. At the same time, components of the sample (e.g., a protein) will also migrate toward the region along the length of the channel where the local pH is the closest to the component's isoelectric point (pI). When, e.g., the protein reaches its pI, it will have a net zero charge and will stop migrating. This step is called “focusing.” An image of the channel after, e.g., all proteins in the sample reach their pIs may show the relative abundance of the various protein species in the sample. The pI of the various protein species can be determined by their relative position to pI markers that are added to the sample solution.

Following the completion of the separation, the focused sample may be re-ionized and transported (e.g., electrokinetically) e.g., for downstream analysis. This step is called “mobilizing” or “mobilization,” and the process must preserve the spatial separation of the focused species. However, during this mobilization, the focused sample must cross a T-junction. This geometry can severely distort the shape of the transiting bands and degrade the spatial resolution between separated protein variants.

### SUMMARY

Disclosed herein are methods, devices, and systems that enable improved quantitative performance for the separation and analysis of analytes in an analyte mixture, with potential applications in biomedical research, clinical diagnostics, and pharmaceutical manufacturing. In particular, narrowing of the width of a tributary channel right where it junctions with the separation channel results in a junction geometry that prevents the spatial distortion of a transiting focused sample.

One aspect of the disclosure is a fluidic device including at least one fluid inlet, at least one fluid outlet, at least one separation channel comprising a first end that is fluidically coupled to the at least one fluid inlet and a second end that is fluidically coupled to the at least one fluid outlet; a first side channel comprising a first portion and a second portion, wherein the first side channel is in fluid communication with the separation channel via the first portion of the first side channel near the second end of the separation channel; wherein a width of the first portion of the first side channel is less than a width of the second portion of the first side channel; and a mobilizer channel in fluid communication with the separation channel near the second end of the separation channel.

In an aspect, the fluidic device further includes a substrate, wherein the substrate comprises a top surface, a bottom surface, and at least one side surface, the separation channel and at least one of the first side channel and mobilizer channel are disposed between the top and bottom surfaces, and wherein the at least one fluid inlet and/or the at least one fluid outlet extends to the at least one side surface, which is not the top or bottom surfaces.

In an aspect, the width of the first portion of the first side channel is equal to or less than 90% of the width of the second portion of the first side channel; alternatively equal to or less than 80% of the width of the second portion of the first side channel; alternatively equal to or less than 70% of the width of the second portion of the first side channel; alternatively equal to or less than 60% of the width of the second portion of the first side channel; alternatively equal to or less than 50% of the width of the second portion of the first side channel; alternatively equal to or less than 40% of the width of the second portion of the first side channel; alternatively equal to or less than 30% of the width of the second portion of the first side channel; alternatively equal to or less than 20% of the width of the second portion of the first side channel; alternatively equal to or less than 10% of the width of the second portion of the first side channel; or alternatively equal to or less than 5% of the width of the second portion of the first side channel.

In an aspect, the width of the first portion of the first side channel is about 600 μm or less, alternatively about 550 μm or less, alternatively about 500 μm or less, alternatively about 450 μm or less, alternatively about 400 μm or less, alternatively about 350 μm or less, alternatively about 300 μm or less, alternatively about 250 μm or less, alternatively about 200 μm or less, alternatively about 150 μm, alternatively about 140 μm, alternatively about 130 μm, alternatively about 120 μm, alternatively about 110 μm, alternatively about 100 μm or less.

In an aspect, the width of the first portion of the first side channel relative to the width of the separation channel is in a ratio of about 3 or less, alternatively a ratio of about 2.5 or less, alternatively about 2 or less, alternatively about 1.5 or less, alternatively about 1 or less.

In an aspect, a length of the second portion of the first side channel is longer than a length of the first portion of the first side channel. In another aspect, the second portion of the first side channel is at least about 5% to about 10,000% longer than the length of the first portion of the first side channel.

In an aspect, the first portion of the first side channel is in fluid communication with the separation channel at a substantially perpendicular angle. In an aspect, the first portion of the first side channel is in fluid communication with the separation channel at an oblique angle. In an aspect, the first side channel is a catholyte channel.

In an aspect, the fluidic device further includes a second side channel, wherein the second side channel is in is in fluid communication with the separation channel near the first end of the separation channel. In an aspect, the second side channel is an anolyte channel.

One aspect of the disclosure is a method including introducing at least one analyte into any fluidic device disclosed; applying an electric field across the separation channel to separate the at least one analyte via isoelectric focusing and generating one or more focused analytes; and mobilizing the one or more focused analytes.

In an aspect, the method further includes imaging the separation channel or a portion thereof during or after the isoelectric focusing separation and/or mobilization.

In an aspect, at least one peak of the at least one analyte detected by the imaging of the separation channel or the portion thereof have a tailing factor (T_{f}) or peak asymmetry factor (Aₛ) of about 1. In another aspect, at least one peak of the at least one or more focused analytes upon mobilizing have a tailing factor (T_{f}) or peak asymmetry factor (Aₛ) of about 1.

In an aspect, the method further includes expelling the one or more mobilized analytes via electrospray ionization into a mass spectrometer from a single orifice.

### BRIEF DESCRIPTION OF THE DRAWINGS

Various aspects of the present disclosure will now be described, by way of example only, with reference to the attached Figures, wherein:

FIG. 1A is a fluidic device according to an aspect of this disclosure. FIG. 1B is an exploded view of where the separation channel and side channel intersect in FIG. 1C is an exploded view of where the separation channel and side channel intersect in a fluidic device where the side channel is a conventional (i.e., not narrowed) width.

FIG. 2 is an exploded view of where the separation channel and side channel intersect according to an aspect of this disclosure.

FIG. 3 is an exploded view of where the separation channel and side channel intersect according to an aspect of this disclosure.

FIGS. 4A and 4B depict the band distortion effect using a conventional fluidic device.

FIGS. 5A and 5B depict the band distortion effect using a fluidic device according to an aspect of this disclosure.

### DETAILED DESCRIPTION

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to aspects of the invention that may afford certain benefits, under certain circumstances. However, other aspects may also be preferred, under the same or other circumstances. Furthermore, the recitation of one or more preferred aspects does not imply that other aspects are not useful and is not intended to exclude other aspects from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different aspects of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same aspects of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, or alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

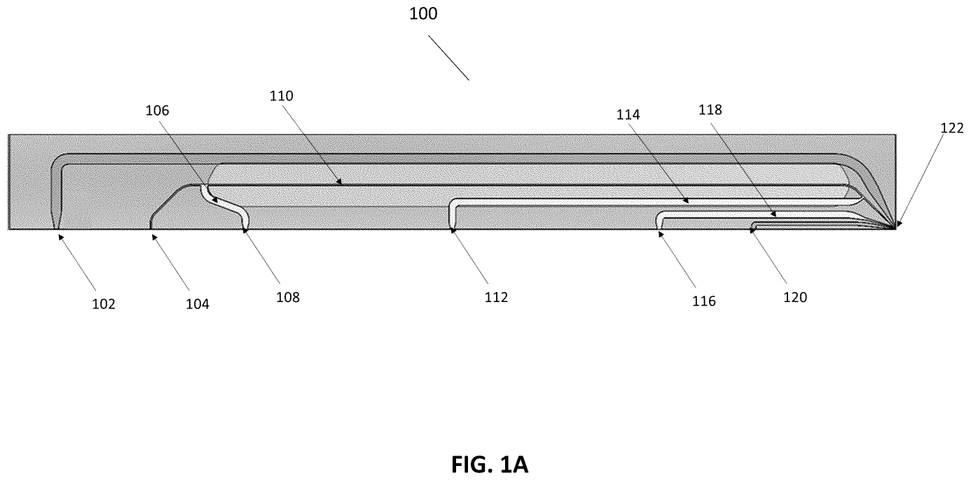

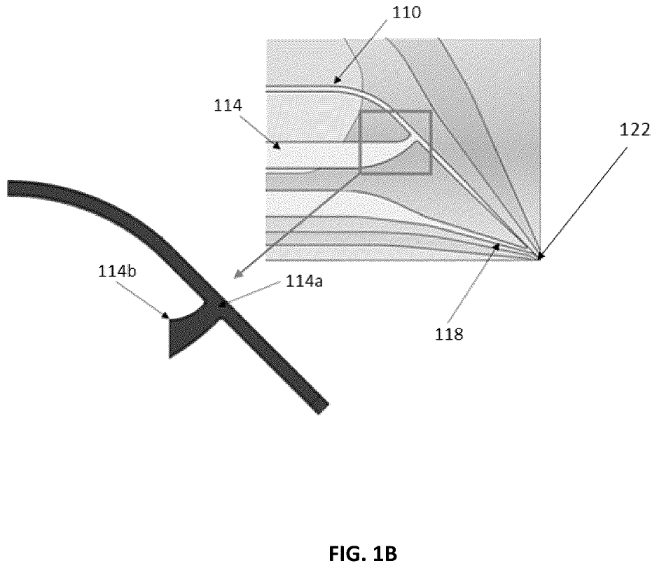

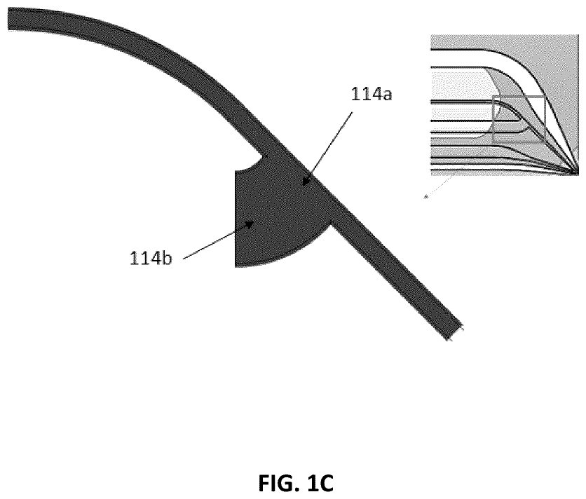

Within the fluidic device, imaged capillary isoelectric focusing (icIEF) may be performed along a separation channel to spatially separate different charge-variant isoforms into distinct “bands”, which can be observed as “peaks” via UV absorption detection. Following the completion of icIEF separation, the focused sample components may be re-ionized and transported (e.g., electrokinetically) toward the chip exit, a process that must preserve the spatial separation of the focused species. During this mobilization, the focused sample may cross an intersection of the separation and other fluid channels (e.g., a T-junction). In certain fluidic devices (e.g., FIG. 1C), the geometry of this intersection can play a significant role in what happens to the sample bands during the transport and certain geometries can severely distort the shape of the transiting bands and degrade the spatial resolution (e.g., between separated protein variants). The disclosed fluidic device has a fluid channel with a narrower portion (e.g., as compared to than what is found in certain other fluidic devices). This results in a geometry that prevents the spatial distortion of transiting focused sample.

FIG. 1A is an example of a fluidic device 100 according to an aspect of the disclosure. In an aspect, the fluidic device may be used in a microfluidic system, e.g., an icIEF system. In an aspect, the fluidic devices includes at least one fluid inlet 104, at least one fluid outlet 122, and at least one separation channel 110. The separation channel has a first end and a second end. In an aspect, the first end of the separation channel is fluidically coupled to the at least one fluid inlet 104 and the second end of the separation channel is fluidically coupled to the at least one fluid outlet 122. In some aspects, the device may comprise a substrate (e.g., made of glass or other material). For example, a substrate may be a glass chip. In some aspects, the substrate comprises a top and bottom surfaces and at least one side surface. In some aspects, the separation channel is disposed between the top and the bottom surfaces. In some aspects, the first side channel and. /or mobilizer channel are disposed between the top and the bottom surfaces. In some aspects, the separation channel and at least one of the first side channel and mobilizer channel are disposed between the top and the bottom surfaces. In some aspects, the at least one fluid inlet and/or the at least one fluid outlet extends to the at least one side surface. In some aspects, the at least one side surface is not the top or bottom surfaces. In some conventional fluidic devices, the inlet and/or outlets may be on the top or bottom surfaces (e.g., above or below the separation channels, above or below the first side channel, above or below mobilizer channel). For example, the inlet and/or outlet may be connected to one or more wells on a top or bottom surfaces. As shown in FIG. 1A, the fluid inlet 104 and/or fluid outlet 122 may extend to the at least one side surface (e.g., which is not the top or bottom surfaces of the substrate of the fluidic device 100. Such inlets and/or outlets may have advantages over the conventional fluidic devices including a reduced service area of the fluidic device and the ability to interface with a mass spectrometer with electrospray ionization.

In some aspects, the fluidic device includes a first side channel 114. As shown in FIG. 1B, the first side channel 114 includes a first portion 114a and a second portion 114b. The first side channel 114 is in fluid communication with the separation channel 110 via the first portion of the first side channel 114a near the second end of the separation channel. In an aspect, the first side channel 114 is a catholyte channel.

In an aspect, the first portion of the first side channel 114a has a width that is less than a width of the second portion of the first side channel 114b (see FIG. 1B). This is distinctly different than other fluidic devices (see, e.g., FIG. 1C), wherein the first portion of the first side channel 114a and the second portion of the first side channel 114b have the same or substantially the same widths.

In an aspect, the width of the first portion of the first side channel is equal to or less than 90% of the width of the second portion of the first side channel. In an aspect, the width of the first portion of the first side channel is equal to or less than 80% of the width of the second portion of the first side channel. In an aspect, the width of the first portion of the first side channel is equal to or less than 70% of the width of the second portion of the first side channel. In an aspect, the width of the first portion of the first side channel is equal to or less than 60% of the width of the second portion of the first side channel. In an aspect, the width of the first portion of the first side channel is equal to or less than 50% of the width of the second portion of the first side channel. In an aspect, the width of the first portion of the first side channel is equal to or less than 40% of the width of the second portion of the first side channel. In an aspect, the width of the first portion of the first side channel is equal to or less than 30% of the width of the second portion of the first side channel. In an aspect, the width of the first portion of the first side channel is equal to or less than 20% of the width of the second portion of the first side channel. In an aspect, the width of the first portion of the first side channel is equal to or less than 10% of the width of the second portion of the first side channel. In an aspect, the width of the first portion of the first side channel is equal to or less than 5% of the width of the second portion of the first side channel. It is appreciated by one of ordinary skill that the width of the first portion of the first side channel may be adjusted to achieve the desired resolution, and the width of the second portion may be adjusted to achieve the desired electrical resistance of the first side channel.

In some aspects, the width of the first portion of the first side channel is adjusted relative to the width of the separation channel. In an aspect, the ratio of the width of the first portion of the first side channel and the width of the separation channel is 3:1 or less. In an aspect, the ratio of the width of the first portion of the first side channel and the width of the separation channel is 2.5:1 or less. In an aspect, the ratio of the width of the first portion of the first side channel and the width of the separation channel is 2:1 or less. In an aspect, the ratio of the width of the first portion of the first side channel and the width of the separation channel is 1.5:1 or less. In an aspect, the ratio of the width of the first portion of the first side channel and the width of the separation channel is 1:1 or less. In some aspects, the separation channel may have a width of 120 μm, and the first portion of the first side channel may have a width between about 120 μm to about 360 μm. In some aspects, the separation channel may have a width of 130 μm, and the first portion of the first side channel may have a width between about 130 μm to about 390 μm. In some aspects, the separation channel may have a width of 140 μm, and the first portion of the first side channel may have a width between about 140 μm to about 420 μm. In some aspects, the separation channel may have a width of 150 μm, and the first portion of the first side channel may have a width between about 150 μm to about 450 μm.

In an aspect, the width of the first portion of the first side channel is about 600 μm or less. In an aspect, the width of the first portion of the first side channel is about 550 μm or less. In an aspect, the width of the first portion of the first side channel is about 500 μm or less. In an aspect, the width of the first portion of the first side channel is about 450 μm or less. In an aspect, the width of the first portion of the first side channel is about 400 μm or less. In an aspect, the width of the first portion of the first side channel is about 350 μm or less. In an aspect, the width of the first portion of the first side channel is about 300 μm or less. In an aspect, the width of the first portion of the first side channel is about 250 μm or less. In an aspect, the width of the first portion of the first side channel is about 200 μm or less. In an aspect, the width of the first portion of the first side channel is about 150 μm or less. In an aspect, the width of the first portion of the first side channel is about 140 μm or less. In an aspect, the width of the first portion of the first side channel is about 130 μm or less. In an aspect, the width of the first portion of the first side channel is about 120 μm or less. In an aspect, the width of the first portion of the first side channel is about 110 μm or less. In an aspect, the width of the first portion of the first side channel is about 100 μm or less.

In some aspects, the second portion of the first side channel is longer than the length of the first portion of the first side channel. In an aspect, the second portion of the first side channel is at least about 5% longer than the length of the first portion of the first side channel. In an aspect, the second portion of the first side channel is at least about 10% longer than the length of the first portion of the first side channel. In an aspect, the second portion of the first side channel is at least about 20% longer than the length of the first portion of the first side channel. In an aspect, the second portion of the first side channel is at least about 30% longer than the length of the first portion of the first side channel. In an aspect, the second portion of the first side channel is at least about 40% longer than the length of the first portion of the first side channel. In an aspect, the second portion of the first side channel is at least about 50% longer than the length of the first portion of the first side channel. In an aspect, the second portion of the first side channel is at least about 60% longer than the length of the first portion of the first side channel. In an aspect, the second portion of the first side channel is at least about 70% longer than the length of the first portion of the first side channel. In an aspect, the second portion of the first side channel is at least about 80% longer than the length of the first portion of the first side channel. In an aspect, the second portion of the first side channel is at least about 90% longer than the length of the first portion of the first side channel. In an aspect, the second portion of the first side channel is at least about 100% longer than the length of the first portion of the first side channel. In an aspect, the second portion of the first side channel is at least about 110% longer than the length of the first portion of the first side channel. In an aspect, the second portion of the first side channel is at least about 120% longer than the length of the first portion of the first side channel. In an aspect, the second portion of the first side channel is at least about 130% longer than the length of the first portion of the first side channel. In an aspect, the second portion of the first side channel is at least about 140% longer than the length of the first portion of the first side channel. In an aspect, the second portion of the first side channel is at least about 150% longer than the length of the first portion of the first side channel. In an aspect, the second portion of the first side channel is at least about 200% longer than the length of the first portion of the first side channel. In an aspect, the second portion of the first side channel is at least about 300% longer than the length of the first portion of the first side channel. In an aspect, the second portion of the first side channel is at least about 400% longer than the length of the first portion of the first side channel. In an aspect, the second portion of the first side channel is at least about 500% longer than the length of the first portion of the first side channel. In an aspect, the second portion of the first side channel is at least about 600% longer than the length of the first portion of the first side channel. In an aspect, the second portion of the first side channel is at least about 700% longer than the length of the first portion of the first side channel. In an aspect, the second portion of the first side channel is at least about 800% longer than the length of the first portion of the first side channel. In an aspect, the second portion of the first side channel is at least about 900% longer than the length of the first portion of the first side channel. In an aspect, the second portion of the first side channel is at least about 1000% longer than the length of the first portion of the first side channel. In an aspect, the second portion of the first side channel is at least about 2000% longer than the length of the first portion of the first side channel. In an aspect, the second portion of the first side channel is at least about 3000% longer than the length of the first portion of the first side channel. In an aspect, the second portion of the first side channel is at least about 4000% longer than the length of the first portion of the first side channel. In an aspect, the second portion of the first side channel is at least about 5000% longer than the length of the first portion of the first side channel. In an aspect, the second portion of the first side channel is at least about 6000% longer than the length of the first portion of the first side channel. In an aspect, the second portion of the first side channel is at least about 7000% longer than the length of the first portion of the first side channel. In an aspect, the second portion of the first side channel is at least about 8000% longer than the length of the first portion of the first side channel. In an aspect, the second portion of the first side channel is at least about 9000% longer than the length of the first portion of the first side channel. In an aspect, the second portion of the first side channel is at least about 10000% longer than the length of the first portion of the first side channel.

In some aspects, the first portion of the first side channel intersects the separation channel at a substantially perpendicular angle, a perpendicular angle, or a right angle. In some aspects, the first portion of the first side channel intersects the separation channel at an oblique angle or a substantially oblique angle.

In some aspects, the fluidic device includes a mobilizer channel 118 that intersects near the second end of the separation channel 110. In an aspect, the fluidic device may further include a second side channel 106. In this aspect, the second side channel 106 is in fluid communication with the separation channel 110 near the first end of the separation channel.

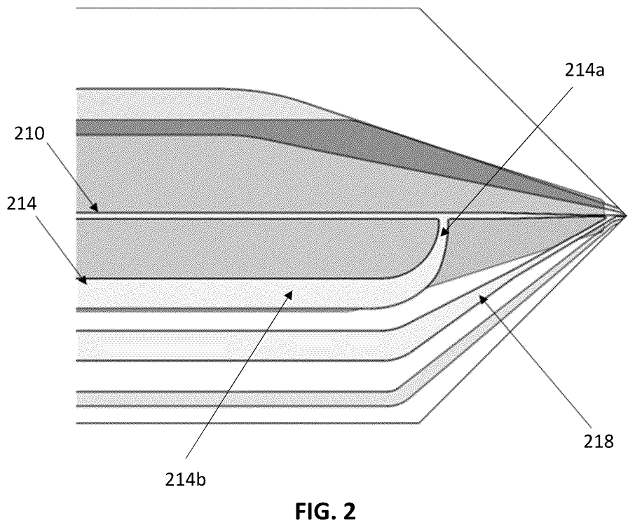

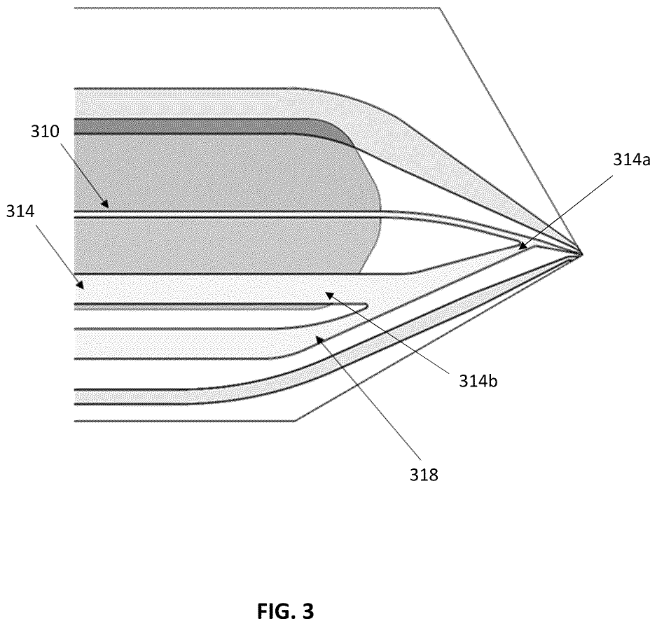

FIG. 2 and FIG. 3 are alternative aspects of the fluidic device. In FIG. 2, the separation channel 210 is in fluidic communication with the first side channel 214 via the first portion of the first side channel 214a near the second end of the separation channel 210. The first portion of the first side channel 214a intersects the separation channel 210 at a substantially perpendicular angle. The width of the first portion of the first side channel 214a is also less than a width of the second portion of the first side channel 214b. The fluidic device also includes a mobilizer channel 218 that intersects near the second end of the separation channel 210.

In FIG. 3, the separation channel 310 is in fluidic communication with the first side channel 314 via the first portion of the first side channel 314a near the second end of the separation channel 310. The first portion of the first side channel 314a intersects the separation channel 310 at a substantially oblique angle. The width of the first portion of the first side channel 314a is also less than a width of the second portion of the first side channel 314b. The fluidic device also includes a mobilizer channel 318 that intersects with the first side channel 314, which then intersects near the second end of the separation channel 310.

### EXAMPLES

These examples are provided for illustrative purposes only and not to limit the scope of the claims provided herein.

### Exemplary Method Using Disclosed Fluidic Device

The disclosed fluidic device 100 may be used in an analytical method. In a non-limiting example, the fluidic device may be used for icIEF or icIEF-MS analysis of at least one analyte. The analyte may be a biomolecule. In non-limiting examples, the biomolecule is a peptide, a polypeptide, a protein, an antibody, a biologic, a biosimilar, or an antibody-drug conjugate. The biomolecule may be intact or hydrolyzed.

In an aspect, the separation channel 110 may be filled with a sample mixture. The sample mixture may include at least one analyte and an amphoteric compound (e.g., ampholyte). The sample mixture is introduced via the fluid inlet 104. The first side channel 114 is filled with a catholyte via the first side channel inlet 112. The second side channel 106 is filled with an anolyte via the second side channel inlet 108. The mobilizer channel 118 is filled with a mobilizer solution via the mobilizer channel inlet 116.

During focusing, an electric potential is applied by an anode electrode in contact with the anolyte. A grounding potential is applied to the opposite end of the separation channel 110 by a first cathode electrode in contact with the catholyte to provide a liquid junction at the intersection of the first side channel 114 and the separation channel 110. Imaging of the separation channel 110 may occur during and/or after the focusing. The resulting focused analyte peaks may have a tailing factor (T_{f}) or peak asymmetry factor (Aₛ) of about 1.

Tris a tailing factor (e, g., the USP coefficient of the peak symmetry). It is calculated using the following equation: T_{f}=(a+b)/2a where a is the distance from the leading edge of the peak to the peak midpoint (perpendicular from the peak highest point) measured at 5% of peak height and b is the distance from the peak midpoint (perpendicular from the peak highest point) to the trailing edge of the peak measured at 5% of peak height. If Tr is about 1, the peak is considered a symmetrical or gaussian peak.

As is calculated with the following equation As =b/a where b is the distance from the peak midpoint (perpendicular from the peak highest point) to the trailing edge of the peak measured at 10% of peak height and a is the distance from the leading edge of the peak to the peak midpoint (perpendicular from the peak highest point) measured at 10% of peak height. If Aₛ>1: tailing and if Aₛ<1: fronting

In some aspects, the images may be used to detect the presence of one or more markers or indicators, e.g., isoelectric point (pI) standards, within the separation channel and thus determine the pIs for the one or more analytes. In some aspects, the images may be used to detect a failure in a separation channel (e.g., bubble formation). In some aspects, data derived from such images may be used to determine when a separation reaction is complete (e.g., by monitoring peak velocities, peak positions, and/or peak widths) and subsequently trigger a mobilization step.

Once focusing is complete, mobilization may be initiated. Imaging of the separation channel 110 may also occur while mobilizing is in process. Imaging of the separation channel 110 may also occur after mobilizing is in process. In some aspects, mobilization may include the introduction of a mobilizer (buffer, acid, and/or electrolyte) from the mobilization channel 118 into the separation channel 110.

In some aspects, the mobilizer may be introduced using hydrodynamic pressure. In some aspects, the mobilizer may be introduced by means of electrophoresis. In some aspects, the mobilizer may be introduced by means of a combination of electrophoresis and hydrodynamic pressure. In some instances, the mobilization of a series of one or more focused analytes may include causing the focused analyte to migrate towards a fluid outlet and/or the second end of the separation channel.

In some instances, the mobilization of a series of one or more focused analytes may include causing the focused analytes to migrate towards a fluid outlet and/or the second end of the separation channel that is in fluid communication with a downstream analytical instrument. In some instances, the outlet or the second end of the separation channel may be in fluid communication with an electrospray ionization (ESI) interface such that the migrating analyte peaks are delivered into a mass spectrometer. In some instances, the image data used to detect analyte peak positions and determine analyte pIs may also be used to correlate analyte separation data with mass spectrometry data. In some instances, the image data used to detect analyte peak positions may be used to yield information on the mobilization reaction and/or to correlate the mobilization information with the mass spectrometry data.

In some instances, at least one analyte may be introduced into a fluidic device (e.g., 100), an electric field is applied across the separation channel 110 to separate the at least one analyte via isoelectric focusing and generate one or more focused analytes. In some instances, mobilizing is performed of the one or more focused analytes. In some instances, imaging is performed of the separation channel 110 or a portion thereof during or after the isoelectric focusing separation and/or mobilization. In some instances, at least one peak of the at least one analyte (detected by the imaging of the separation channel 110 or the portion thereof) has a tailing factor (T_{f}) or peak asymmetry factor (Aₛ) of about 1. In some instances, at least one peak of the at least one or more focused analytes upon mobilizing have a tailing factor (T_{f}) or peak asymmetry factor (Aₛ) of about 1.

A nebulizing gas may be supplied through gas inlets 102, 120 to provide a nebulizing gas flow about the fluid emission from the outlet 122.

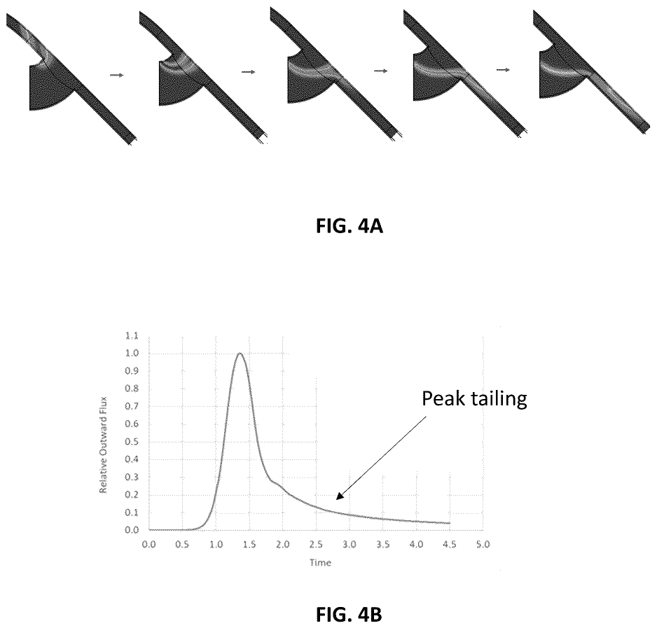

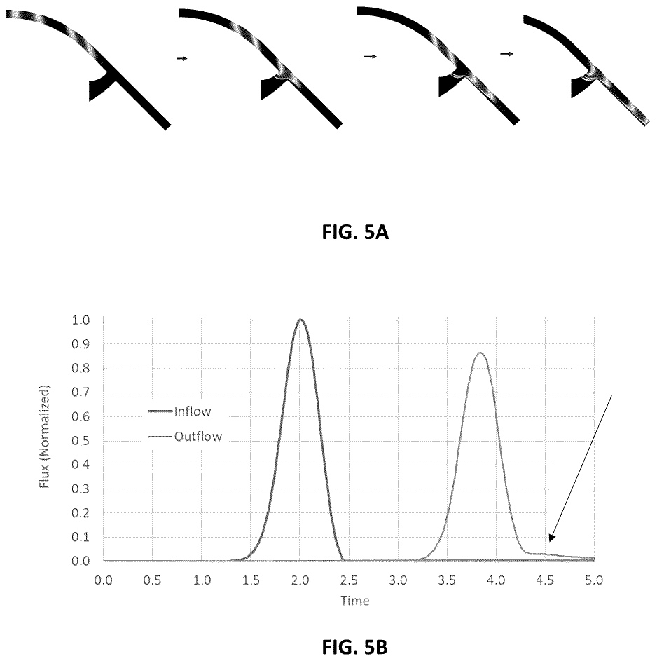

As shown in FIG. 4A, in some fluidic devices, the mobilized band is stretched and distorted at the junction between the catholyte channel and the separation channel (e.g., during mobilization). This may be partly due to a sharp drop in electric field strength and/or distortion of field lines at catholyte junction, which may lead to peak tailing, as shown in FIG. 4B. As shown in FIG. 5A, narrowing of, e.g., a portion of a catholyte channel reduces the band distortion. The narrowing near the junction area may be associated with a milder electric field disruption, resulting in less peak tailing (see, e.g., FIG. 5B).

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A fluidic device comprising:
at least one fluid inlet (104);
at least one fluid outlet (122);
at least one separation channel (110) comprising a first end that is fluidically coupled to the at least one fluid inlet and a second end that is fluidically coupled to the at least one fluid outlet;
a first side channel (114) comprising a first portion (114a) and a second portion (114b),
wherein the first side channel is in fluid communication with the separation channel via the first portion of the first side channel near the second end of the separation channel;
wherein a width of the first portion of the first side channel is less than a width of the second portion of the first side channel; and
a mobilizer channel (118) in fluid communication with the separation channel near the second end of the separation channel.

2. The fluidic device of claim 1 comprising a substrate, wherein:
the substrate comprises a top surface, a bottom surface, and at least one side surface,
the separation channel and at least one of the first side channel and mobilizer channel are disposed between the top and bottom surfaces, and wherein
the at least one fluid inlet and/or the at least one fluid outlet extends to the at least one side surface, which is not the top or bottom surfaces.

3. The fluidic device of claim 1, wherein the width of the first portion of the first side channel is equal to or less than 90% of the width of the second portion of the first side channel; alternatively equal to or less than 80% of the width of the second portion of the first side channel; alternatively equal to or less than 70% of the width of the second portion of the first side channel; alternatively equal to or less than 60% of the width of the second portion of the first side channel; alternatively equal to or less than 50% of the width of the second portion of the first side channel; alternatively equal to or less than 40% of the width of the second portion of the first side channel; alternatively equal to or less than 30% of the width of the second portion of the first side channel; alternatively equal to or less than 20% of the width of the second portion of the first side channel; alternatively equal to or less than 10% of the width of the second portion of the first side channel; or alternatively equal to or less than 5% of the width of the second portion of the first side channel.

4. The fluidic device of claim 3, wherein the width of the first portion of the first side channel is about 600 μm or less, alternatively about 550 μm or less, alternatively about 500 μm or less, alternatively about 450 μm or less, alternatively about 400 μm or less, alternatively about 350 μm or less, alternatively about 300 μm or less, alternatively about 250 μm or less, alternatively about 200 μm or less, alternatively about 150 μm, alternatively about 140 μm, alternatively about 130 μm, alternatively about 120 μm, alternatively about 110 μm, alternatively about 100 μm or less.

5. The fluidic device of claim 1, wherein the width of the first portion of the first side channel relative to the width of the separation channel is in a ratio of about 3 or less, alternatively a ratio of about 2.5 or less, alternatively about 2 or less, alternatively about 1.5 or less, alternatively about 1 or less.

6. The fluidic device of claim 1, wherein a length of the second portion of the first side channel is longer than a length of the first portion of the first side channel.

7. The fluidic device of claim 6, wherein the second portion of the first side channel is at least about 5% to about 10,000% longer than the length of the first portion of the first side channel.

8. The fluidic device of claim 1, wherein the first portion of the first side channel is in fluid communication with the separation channel at a substantially perpendicular angle.

9. The fluidic device of claim 1, wherein the first portion of the first side channel is in fluid communication with the separation channel at an oblique angle.

10. The fluidic device of claim 1, wherein the first side channel is a catholyte channel.

11. The fluidic device of claim 1, further comprising a second side channel, wherein the second side channel is in is in fluid communication with the separation channel near the first end of the separation channel.

12. The fluidic device of claim 11, wherein the second side channel is an anolyte channel.

13. A method comprising:
introducing at least one analyte into the fluidic device of claim 1;
applying an electric field across the separation channel to separate the at least one analyte via isoelectric focusing and generating one or more focused analytes; and
mobilizing the one or more focused analytes.

14. The method of claim 13, further comprising imaging the separation channel or a portion thereof during or after the isoelectric focusing separation and/or mobilization.

15. The method of claim 14, wherein at least one peak of the at least one analyte detected by the imaging of the separation channel or the portion thereof have a tailing factor (T_{f}) or peak asymmetry factor (Aₛ) of about 1.

16. The method of claim 15, wherein at least one peak of the at least one or more focused analytes upon mobilizing have a tailing factor (T_{f}) or peak asymmetry factor (Aₛ) of about 1.

17. The method of claim 14, further comprising expelling the one or more mobilized analytes via electrospray ionization into a mass spectrometer from a single orifice.
